(19) United States
(12) Patent Application Publication
(10) Pub. No.: US 2013/0071330 A1
(43) Pub. Date: Mar. 21, 2013

(54) METHODS OF IDENTIFYING AGENTS EFFECTIVE TO TREAT COGNITIVE DECLINE AND DISEASES ASSOCIATED THEREWITH

(71) Applicant: Susan Catalano, Pittsburgh, PA (US)

(72) Inventor: Susan Catalano, Pittsburgh, PA (US)

(21) Appl. No.: 13/581,144

(22) PCT Filed: Feb. 28, 2011
(86) PCT No.: PCT/US11/26530

§ 371 Date: Dec. 7, 2012

Related U.S. Application Data
(60) Provisional application No. 61/308,719, filed on Feb. 26, 2010.
(60) Provisional application No. 61/309,099, filed on Mar. 1, 2010.

(51) Int. Cl. C12Q 1/02; A61K 49/00

(52) U.S. Cl. CPC C12Q 1/025; A61K 49/00
USPC 424/9.2; 435/29; 435/7.1

(57) ABSTRACT

Embodiments described herein are directed to methods of identifying agents effective to treat cognitive decline and diseases associated therewith. Embodiments described herein are also directed to kits for performing methods described herein. Embodiments described are also directed to methods of diagnosis and uses of the same.

### CROSS-REFERENCE TO RELATED APPLICATIONS

This application claims the benefit under 35 U.S.C. §119(e) to U.S. Provisional Appln. No. 61/308,719, filed Feb. 26, 2010, and to U.S. Provisional Appln. No. 61/309,099, filed Mar. 1, 2010, the contents of which are incorporated by reference herein as if set forth in their entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

Not applicable

### BRIEF SUMMARY OF THE INVENTION

In some embodiments, the present invention provides methods of identifying an agent effective to treat cognitive decline. In some embodiments, the methods comprise contacting a test agent and a processed product of amyloid precursor protein with a composition comprising at least one neuronal cell and measuring a processed product of amyloid precursor protein-mediated effect on the at least one neuronal cell, wherein a test agent that inhibits the processed product of amyloid precursor protein-mediated effect on the cell is identified as an agent that is effective to treat cognitive decline. In some embodiments, the processed product of amyloid precursor protein-mediated effect on the cell is an effect on membrane trafficking. In some embodiments, the processed product of amyloid precursor protein is an Amyloid beta (Abeta) monomer, Abeta oligomer, or combinations thereof.

In some embodiments, the test agent (also sometimes referred to as “test compound” herein) is an agent effective to treat cognitive decline and the test agent does not significantly affect membrane trafficking in the absence of the Abeta monomer, Abeta oligomer, or combinations thereof. In some embodiments, the processed product of amyloid precursor protein is contacted with the cell at a sublethal concentration.

In some embodiments, the method comprises contacting a negative control or a positive control with a second composition of cells and measuring the processed product of amyloid precursor protein-mediated effect on the composition of cells. In some embodiments, the inhibition of the processed product of amyloid precursor protein-mediated effect on the composition of cells by the test agent is compared to the mediated effect in the presence or absence of the negative control or positive control. In some embodiments, the methods further comprise contacting a test agent identified as an agent effective to treat cognitive decline with an animal and confirming that the test agent is an agent effective to treat cognitive decline in the animal. In some embodiments the animal is an animal with an Abeta-induced fear condition deficit model, wherein a test agent that inhibits the effects of Abeta in the model is confirmed to be an agent effective to treat cognitive decline.

In some embodiments, the present invention provides methods of identifying an agent effective to treat a neurodegenerative or psychiatric disease, wherein a symptom of the disease is cognitive decline, comprising contacting a test agent and a processed product of amyloid precursor protein with a composition comprising at least one neuronal cell and measuring a processed product of amyloid precursor protein-mediated effect on the at least one neuronal cell, wherein a test agent that inhibits the processed product of amyloid precursor protein-mediated effect on the cell is identified as an agent effective to treat the neurodegenerative or psychiatric disease.

In some embodiments, the methods identify an agent effective to treat Alzheimer's Disease.

In some embodiments, the present invention provides agents identified by the methods disclosed herein. In some embodiments, the present invention provides pharmaceutical compositions comprising an agent identified by a method disclosed herein.

In some embodiments, the present invention provides kits for identifying an agent effective to treat cognitive decline. In some embodiments, the kit comprises at least one of a test agent; a negative control; a positive control; a neuronal cell; a processed product of amyloid precursor protein; or instructions on how to identify an agent effective to treat cognitive decline. In some embodiments, the kit comprises an Abeta monomer, oligomer, or combinations thereof.

In some embodiments, the present invention provides methods of diagnosing an animal with cognitive decline or a disease associated therewith. In some embodiments, the methods comprise contacting a sample from an individual with a composition comprising at least one neuronal cell and measuring a membrane trafficking effect of the cell. In some embodiments, a change in membrane trafficking indicates that the animal has cognitive decline or a disease associated therewith. In some embodiments, the disease that is diagnosed is Alzheimer's Disease (also sometimes referred to as “AD” herein). In some embodiments, the sample is cerebrospinal fluid (CSF), blood, plasma, or any combination thereof. In some embodiments, the present invention provides kits for diagnosing an animal with cognitive decline or a disease associated therewith comprising at least one of: instructions for diagnosing said animal; a negative control; a positive control; a neuronal cell; or a yellow tetrazolium salt. In some embodiments, the kit comprises a negative control, wherein the negative control is a sample from an animal that is not afflicted with cognitive decline or a disease associated therewith.

In some embodiments, the present invention provides methods of identifying an Abeta binding partner.

### DESCRIPTION OF DRAWINGS

FIG. 1 shows results of an MTT assay in the presence and absence of a processed product of amyloid precursor protein.

FIG. 2 shows inhibition of processed product of amyloid precursor protein-mediated membrane trafficking effect by Compound Z.

FIG. 3 shows Compound Z inhibiting the memory loss effects of a processed product of amyloid precursor protein.

FIG. 4 shows Compound Z inhibiting the membrane trafficking effects of Abeta assemblies isolated from AD patients.

### DETAILED DESCRIPTION

Before the compositions and methods are described herein, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention. Unless defined otherwise, all technical and scientific terms used herein have the same meaning as commonly understood by one of ordinary skill in the art. All publications mentioned herein are incorporated by reference in their entirety. Nothing herein is to be construed as an admission that the embodiments of the invention described herein are not entitled to antedate any reference disclosed herein by virtue of prior invention.

It must also be noted that as used herein the singular forms “a”, “an”, and “the” include plural reference unless the context clearly dictates otherwise.

As used herein, the term “about” means plus or minus about 10% of the numerical value of the number with which it is being used. For example, about 50% means in the range of about 45%-55%.

“Administering” when used in conjunction with a therapeutic means to administer a therapeutic directly into or onto a target tissue or to administer a therapeutic to an individual, whereby the therapeutic positively impacts the tissue to which it is targeted. Therefore, “administering,” when used in conjunction with an agent, can include, but is not limited to, providing an agent to a subject systemically by, for example, intravenous injection, whereby the therapeutic reaches the target tissue or cell. A composition may be also administered in a standard manner such as orally, parenterally, transmucosally (e.g., sublingually or via buccal administration), topically, transdermally, rectally, via inhalation (e.g., nasal or deep lung inhalation). Parenteral administration includes, but is not limited to intravenous, intraarterial, intraperitoneal, subcutaneous, and intramuscular. Administration can also be combined with other methods, which include, but are not limited to, heating, radiation, ultrasound and the use of delivery agents.

The term “animal” as used herein includes, but is not limited to, humans and non-human vertebrates such as wild, domestic and farm animals.

The term “improves” is used to convey that the present invention changes either the characteristics and/or the physical attributes of the object to which it is being provided, applied or administered. The term “improves” may also be used in conjunction with a diseased state such that when a diseased state is “improved” the symptoms or physical characteristics associated with the diseased state are diminished, reduced or eliminated.

The term “inhibiting” includes the administration of a compound of the present invention to prevent the onset of the symptoms, alleviating the symptoms, or eliminating the disease, condition or disorder.

By “pharmaceutically acceptable”, it is meant the carrier, diluent or excipient must be compatible with the other ingredients of the formulation and not deleterious to the recipient thereof.

As used herein, the term “therapeutic” means an agent utilized to treat, combat, ameliorate, prevent or improve an unwanted condition or disease of a patient. In some embodiments, the agents identified by the methods disclosed herein can be used to treat cognitive decline and diseases associated with cognitive decline. Examples of diseases associated with cognitive decline include, but are not limited to, neurodegenerative disease and psychiatric diseases. For example, Alzheimer's Disease may be treated with the agents identified using the methods described herein.

A “therapeutically effective amount” or “effective amount” of a composition is an amount that is determined and used to achieve a desired effect, e.g, inhibit, block, or treat cognitive decline. The activity contemplated by the agents identified by the present methods includes both medical therapeutic and/or prophylactic treatment, as appropriate. The specific dose of a compound administered according to obtain therapeutic and/or prophylactic effects can be determined and will depend upon the particular circumstances surrounding the case, including, for example, the agent administered, the route of administration, and the condition being treated. However, it will be understood that the effective amount administered can be determined by a physician in the light of the relevant circumstances including the condition to be treated, the choice of compound to be administered, and the chosen route of administration; and therefore, the above dosage ranges are not intended to limit the scope of the invention in any way. A therapeutically effective amount of a compound or agent of this invention is typically an amount such that when it is administered it is sufficient to achieve an effective systemic concentration or local concentration in the tissue or cells.

The terms “treat”, “treated”, or “treating” as used herein may refer to both therapeutic treatment and/or prophylactic or preventative measures, wherein the object is to prevent or slow down (lessen) an undesired physiological condition, disorder or disease, or to obtain beneficial or desired clinical results. Beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment can also include eliciting a clinically significant response without excessive levels of side effects. Treatment can also include prolonging survival as compared to expected survival if not receiving treatment.

As used herein, the term “contacting” refers to the bringing together or combining of molecules such that they are within a distance for allowing of intermolecular interactions such as the non-covalent interaction between a two peptides or one protein and an agent. In some embodiments, contacting occurs in solution phase in which the combined or contacted molecules are dissolved in a common solvent and are allowed to freely associate. In some embodiments, the contacting can occur within a cell or in a cell-free environment. In some embodiments, the cell-free environment is the lysate produce from a cell. In some embodiments, a cell lysate may be a whole-cell lysate, nuclear lysate, cytoplasm lysate, and combinations thereof. In some embodiments, the cell-free lysate is only lysate obtained from a nuclear extraction and isolation wherein the nuclei of a cell population are removed from the cells and then lysed. In some embodiments, the nuclei are not lysed, but are still considered to be a cell-free environment. The agents can also be mixed such as through vortexing, shaking, and the like.

Human amyloid β is a cleavage product of an integral membrane protein, amyloid precursor protein (APP), found concentrated in the synapses of neurons. Amyloid β oligomers have been demonstrated to cause Alzheimer's Disease by inducing changes in neuronal synapses that block learning and memory. Amyloid β fibrils have been associated with the advanced stages of Alzheimer's Disease. However, very little is known about the intracellular or extracellular location of oligomer formation and the structural state of the oligomer. For example, the number of amyloid β subunits that associate to form the oligomer is currently unknown, as is the structural form of the oligomers, which residues are exposed, and whether more than one structural state of oligomer is neuroactive.

Prior to the present invention, methods of identifying agents that can be used to treat the effects of Amyloid β in vitro have not been able to predict whether the agents would also work in vivo. Therefore, there has been a long-felt and unmet need for methods of identifying agents that are effective to treat cognitive decline and its associated diseases in an individual or animal. The present invention fulfills this need as well as others.

In some embodiments, the present invention provides methods of identifying an agent effective to treat cognitive decline. In some embodiments, the present invention provides methods of identifying an agent effective to treat a disease associated with cognitive decline.

As used herein, the term “agent” refers to any compound or composition that can be tested in the methods described herein. Examples of agents that can be used include, but are not limited to, a small organic molecule, an antibody, antibody fragment, siRNA, nucleic acid molecule (RNA or DNA), antisense oligonucleotide, peptide, peptide mimetic, and the like. In some embodiments, an agent can be isolated or not isolated. An agent can be a library of agents. The library can be homogeneous or heterogeneous for the class of molecule within the library. As a non-limiting example, an agent can be a library of agents that are used in the present methods to identify an agent effective to treat cognitive decline, diseases associated with cognitive decline, or Alzheimer's Disease. If a mixture of agents is found to be effective, the pool can then be further purified into separate components to determine which components are in fact effective.

In some embodiments, the methods comprise contacting a test agent and a processed product of amyloid precursor protein with a composition comprising at least one cell. In some embodiments, the cell is a neuronal cell. In some embodiments, the method also comprises measuring a processed product of amyloid precursor protein-mediated effect on the at least one cell.

A “processed product of amyloid precursor protein-mediated effect” refers to an effect on a cell that is contacted with a processed product of amyloid precursor protein. For example, it has been found that when a neuronal cell is contacted with an Amyloid β (“Abeta”) oligomer relative rates of membrane trafficking are modulated. This inhibition can be visualized with many assays, including but not limited to, an MTT assay. For example, yellow tetrazolium salts are endocytosed by cells and the salts are reduced to insoluble purple formazan in the endosomal pathway. The level of purple formazan is a reflection of the number of actively metabolizing cells in culture, and reduction in the amount of formazan is taken as a measure of cell death or metabolic toxicity in culture. When cells that are contacted with a yellow tetrazolium salt are observed through a microscope, the purple formazan is first visible in intracellular vesicles that fill the cell. Over time, the vesicles are exocytosed and the formazan precipitates as needle-shaped crystals on the outer surface of the plasma membrane as the insoluble formazan is exposed to the aqueous media environment.

For example, cells respond to sublethal concentrations of Abeta oligomers by accelerating exocytosis, accelerating endocytosis, inhibiting exocytosis, or inhibiting endocytosis. In some embodiments, the rate of exocytosis and/or endocytosis remain the same in the presence of Abeta oligomers. In some embodiments, Abeta oligomers accelerate the exocytosis rate of reduced formazan, while leaving the endocytosis rate unaffected or vice versa. These observations can, in some embodiments, can also be observed in mature primary neurons in vitro. These observations (e.g. morphological shifts due to differences in membrane trafficking) may also be quantified via automated microscopy and image processing. For example, in some embodiments, images may be captured and analyzed with the Cellomics VTI automated microscope platform, using, for example, the Neuronal Profiling algorithm. For statistical analysis, a Tukey-Kramer pair-wise comparison with unequal variance may be used. When neuronal cells are treated with Abeta oligomers there may be no overall change in the total amount of reduced formazan, but rather a shift in its morphology. Accordingly, in some embodiments, a “processed product of amyloid precursor protein-mediated effect” can refer to a shift in cell morphology or quantifying the amount of reduced formazan inside, outside, and/or on the cell. In some embodiments, a processed product of amyloid precursor protein-mediated effect is the change in concentration of a salt in the cell or in the medium outside the cell.

In some embodiments, a test agent that inhibits the processed product of amyloid precursor protein-mediated effect on the cell is identified as an agent that is effective to treat cognitive decline, a disease or condition associated with cognitive decline (e.g. Alzheimer's Disease). In some embodiments, the test agent inhibits the binding of the processed product to the cell. In some embodiments, the test agent inhibit the membrane trafficking effect of a processed product of amyloid precursor protein. In some embodiments, the test agent inhibits the processed product of amyloid precursor protein-mediated effect on the trafficking (e.g. endocytosis rate or exocytosis rate) of reduced formazan.

In some embodiments, the effect of the test agent is compared to a negative or positive control. A “negative control,” as used herein, refers to an agent that does not inhibit, enhance, or have a significant effect on a processed product of amyloid precursor protein-mediated effect. A “negative control” can be another compound or the negative control can be the vehicle that is used to carry or solubilize the test agent. For example, in some embodiments, the negative control is water, DMSO, and the like.

A “positive control,” as used herein, refers to an agent that can inhibit or enhance a processed product of amyloid precursor protein-mediated effect. For example, memantine can be used as a positive control. In some embodiments, memantine can be used to inhibit a processed product of amyloid precursor protein-mediated effect. In some embodiments, Compound Z, which has a structure of the formula:

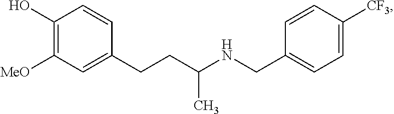 [SMILES: COc1cc(CCC(C)NCc2ccc(C(F)(F)F)cc2)ccc1O]

can be used as a positive control. Compound Z may also be referred to as CT0109.

In some embodiments, a test agent is said to be effective to treat cognitive decline or a disease associated therewith when it can inhibit a processed product of amyloid precursor protein-mediated effect more than about 2, 3, 4, 5, 6, 7, 8, 9, or 10 fold as compared to a negative control. In some embodiments, a test agent is said to be effective when it can inhibit a processed product of amyloid precursor protein-mediated effect more than 2, 3, 4, 5, 6, 7, 8, 9, or 10 fold as compared to a positive control.

In some embodiments, to compare a test agent's effects on a composition of cells to a negative or positive control's effect on cells both the test agent and the control (e.g. positive or negative) is contacted with a composition of cells. The composition of cells are different populations, that is, in some embodiments, the test agent and the control are not contacted with the same cells. The effects of the test agents and the controls are determined and then compared. The effects of the test agent are compared to the effects of the controls to determine if the test agent has a greater, lesser, or similar effect as the control.

As discussed herein, a processed product of amyloid precursor protein (APP) is product that results from the processing of APP. In some embodiments, the processed product is an amyloid β (“Abeta”) oligomer. In some embodiments, the processed product is an Abeta monomer.

The monomers or oligomers of amyloid β may be obtained from any source. For example, in some embodiments, commercially available amyloid β monomers and/or amyloid β oligomers may be used, and in other embodiments, amyloid β monomers and/or amyloid β oligomers that are used can be isolated and purified from animal or human dementia patient by the skilled artisan using any number of known techniques. In general, the amyloid β monomers and/or amyloid β oligomers used herein and amyloid β of various embodiments may be soluble in an aqueous solution.

The amyloid β (“Abeta”) contacted with a composition of cells may be of any isoform. For example, in some embodiments, the amyloid β monomers may be amyloid β 1-42, and in other embodiments the amyloid β monomers may be amyloid β 1-40 or. In still other embodiments, the amyloid β may be amyloid β 1-39 or amyloid β 1-41. Hence, the amyloid β of various embodiments may encompass any C-terminal isoform of amyloid β. Yet other embodiments include amyloid β in which the N-terminus has been truncated, and in some embodiments, the N-terminus of any of amyloid β C-terminal isomers described above may be truncated to amino acid residue number 2, 3, 4, 5, or 6. For example, amyloid β may encompass amyloid β 1-42, amyloid β 2-42, amyloid β 3-42, amyloid β 4-42, or amyloid β 5-42 and mixtures thereof. In some embodiments, amyloid β may comprise amyloid β 1-40, amyloid β 2-40, amyloid β 3-40, amyloid β 4-40, or amyloid β 5-40.

The amyloid β used in various embodiments may be wild type, i.e. having an amino acid sequence that is identical to the amino acid sequence of amyloid β synthesized in vivo by the majority of the population, or in some embodiments, the amyloid β may be a mutant amyloid β. Embodiments are not limited to any particular variety of mutant amyloid β. For example, in some embodiments, the amyloid β contacted with the composition comprising at least one neuronal cell may include a known mutation, such as, for example, amyloid β having the “Dutch” (E22Q) mutation or the “Arctic” (E22G) mutation. Such mutated monomers may include naturally occurring mutations such as, for example, forms of amyloid β isolated from populations of individuals that are predisposed to, for example, Alzheimer's Disease, familial forms of amyloid β. In other embodiments, mutant amyloid β monomers may be synthetically produced by using molecular techniques to produce an amyloid β mutant with a specific mutation. In still other embodiments, mutant amyloid β monomers may include previously unidentified mutations such as, for example, those mutants found in randomly generated amyloid β mutants. The term “amyloid β” as used herein is meant to encompass both wild type forms of amyloid β as well as any of the mutant forms of amyloid β.

In some embodiments, the amyloid β may be of a single isoform. In other embodiments, various C-terminal isoforms of amyloid β and/or various N-terminal isoforms of amyloid β may be combined to form amyloid β mixtures that can be contacted with a composition comprising at least one neuronal cell. In yet other embodiments, the amyloid β may be derived from amyloid precursor protein (APP) that is contacted with the composition comprising at least one neuronal cell and is cleaved in situ, and such embodiments, various isoforms of amyloid β may be contacted with the cell. Truncation of the N-terminus and/or removal of C-terminal amino acids may occur within the cell, within the media of that the cell is grown in and after amyloid β has been added. Therefore, in the methods described herein a processed product of amyloid precursor protein may include a variety of amyloid β isoforms even when a single isoform is initially contacted with the neuronal cell.

The amyloid β used in various embodiments may be derived from any source. For example, in some embodiments, the amyloid β monomers may be isolated from a natural source such as living tissue, and in other embodiments, the amyloid β may be derived from a synthetic source such as transgenic mice or cultured cells. In certain embodiments, amyloid β monomers or amyloid β oligomers may be derived from a source expressing a single form of amyloid β, and in other embodiments, a combination of amyloid β monomers and amyloid β oligomers may be expressed in a single source to produce oligomers that contain more than one form of amyloid β. In still other embodiments, amyloid β monomers and amyloid β oligomers may be derived from different sources, and in such embodiments, the individual sources may produce the same or different forms of amyloid β monomer. It is noted that various isoforms of each type or form of amyloid β monomer may be included in every collection of amyloid β monomers, as N-terminal and C-terminal truncations may occur before the amyloid β is contacted with the cell regardless of the source or form of amyloid β monomer used. Additionally, amyloid β oligomers used and/or the oligomers that are assembled may be homo-oligomers, which contain a single isoform of amyloid β, or hetero-oligomers, which contain more than one isoform of amyloid β, for example 1-40 and/or 1-42 monomers.

In some embodiments, the amyloid β monomers, oligomers, or combinations thereof or the processed product of APP are isolated from normal and/or patients that have been diagnosed with cognitive decline or diseases associated therewith, such as, but not limited to, Alzheimer's Disease. In some embodiments, the amyloid β monomers, oligomers, or combinations thereof or the processed product of APP are Abeta assemblies that have been isolated from normal or diseased (e.g. Alzheimer's Disease) patients. In some embodiments, the Abeta assemblies are high molecular weight, e.g. greater than 100 KDa. In some embodiments, the Abeta assemblies are intermediate molecular weight, e.g. 10 to 100 KDa. In some embodiments, the Abeta assemblies are less than 10 KDa.

In some embodiments, the processed product of amyloid precursor protein can be contacted with a composition comprising at least one neuronal cell in the absence of the test agent. The processed product of amyloid precursor protein-mediated effect on the cell can be determined. The same or different cell then can be contacted as described herein with the test agent in the absence or presence of a processed product of amyloid precursor protein and it can be determined whether or not the test agent can modulate (e.g. inhibit or enhance) the processed protein's mediated effect on the cell. A test agent that inhibits the mediated effect is said to be effective to treat cognitive decline or a disease associated therewith.

As used herein, the term “composition comprising at least one neuronal cell” can be used to refer to a population of cells. In some embodiments, the neuronal cell is a primary neuronal cell. In some embodiments, the neuronal cell is an immortalized or transformed neuronal cell. A primary neuronal cell is a neuronal cell that can differentiate into other types of neuronal cells, such as glia cells. In some embodiments, the composition comprising at least one neuronal cell is free of glia cells. In some embodiments, the composition comprises less than about 30%, 25%, 20%, 15%, 10%, 5%, or 1% of glia cells. The primary neuronal cell can be derived from any area of the brain of an animal. In some embodiments, the neuronal cell is a hippocampal cell. The presence of glia cells can be determined by any method. In some embodiments, glia cells are detected by the presence of GFAP and neurons can be detected by staining positively with antibodies directed against MAP2.

In some embodiments, the test agent and the processed product of amyloid precursor protein are contacted with the composition comprising at least one neuronal cell in vitro. In some embodiments, the test agent and the processed product are contacted with cell simultaneously. In some embodiments, the test agent is contacted with the cell prior to the processed product being contacted with the cell. In some embodiments, the test agent is contacted with the cell after the processed product has been contacted with the cell.

In some embodiments, the test agent that is identified to be an agent that is effective to treat cognitive decline, diseases associated with cognitive decline (e.g. Alzheimer's Disease) is also an agent that does not significantly affect membrane trafficking in the absence of the processed product of amyloid precursor protein. In some embodiments, the test agent has no effect on membrane trafficking in the absence of the processed product of amyloid precursor protein.

As discussed herein, measuring the processed product of amyloid precursor protein-mediated effect may include measuring membrane trafficking. Measuring membrane trafficking may include measuring exocytosis of a compound out of the cell and/or measuring endocytosis of a compound into a cell.

The processed product of amyloid precursor protein can be contacted with the composition comprising at least one neuronal cell at any concentration desired. In some embodiments, the concentration is a sublethal concentration. In some embodiments, the term “sublethal concentration” refers to a concentration that will not kill more than 50% of a population cells in vitro. In some embodiments, the sublethal concentration kills less than 50%, 40%, 30%, 20%, 10%, 5% 1%, 0.5%, or 0.1% of a cell population. In some embodiments, a sublethal concentration will not kill any cells. Cell death can be measured by any method. For example, cell death can be monitored by visualizing DNA fragmentation. DNA fragmentation is a phenotype seen when cells are undergoing apoptosis. For example, cell death can be monitored by visualizing cell nuclei with DAPI staining. DAPI stains DNA and can show DNA fragmentation, a hallmark of apoptosis. Cell death can also occur via necrosis, which can also be measured and quantified.

In some embodiments, a sublethal concentration can be synaptotoxic. In some embodiments, a sublethal concentration is a concentration that does not effect the synaptotoxicity of a neuron or group of neuronal cells. An agent is synaptotoxic if the agent alters the synaptic function as compared to the synaptic function in the absence of the agent. A change in synaptic function can be either a gain or loss in the number of synapses formed in vitro or in vivo. Synaptotoxicity can also be measured by measuring neurotransmitter release by the neuronal cells. If an amount of a neurotransmitter is changed in the presence of an agent then it can be said to have be synaptotoxic. In some embodiments, the sublethal concentration reduces the number of synapses by less than 50%, 40%, 30%, 20%, 10%, 5% 1%, 0.5%, or 0.1% when compared to the number of synapses in the absence of the agent. In some embodiments, the sublethal concentration does not reduce the number of synapses.

As described herein, the test agent is contacted with a composition comprising at least one neuronal cell. In some embodiments, the neuronal cell is grown for at least about 7, 14, 15, 16, 17, 18, 19, 20 or 21 days prior to being contacted with the test agent. The neuronal cell can be grown in vitro prior to being contacted with a test agent.

As described herein, the methods can be used to identify agents that can be used to treat cognitive decline or diseases associated with cognitive decline. These methods are based upon the surprising discovery that the in vitro assays described herein can be used to predict in vivo efficacy. The in vivo efficacy may not be completely curative. That is the effect of a compound identified as being effective using the in vitro methods may not completely inhibit cognitive decline. To be effective in vivo the test agent that is identified as an agent effective to inhibit cognitive decline or a disease associated therewith is effective if, for example, the agent slows the rate of cognitive decline. For example, an animal's or individual's rate cognitive decline will be slowed, reduced or completely inhibited when contacted with the agent identified with the methods described herein. These methods have the surprising result of being able to predict in vivo efficacy, which leads to an increase in efficiency in drug identification and a decrease in spending on therapeutic candidates that will not have in vivo efficacy. Agents that may work in other in vitro assays but not in the present methods disclosed herein would not be investigated in vivo, which saves money and time. The presently described invention also surprisingly enables one of skill in the art to optimize a lead candidate based upon an in vitro screening method prior to the initiation of Phase 1 clinical study or prior to market introduction as a dietary supplement or food product.

In some embodiments, the methods described herein further comprise contacting a test agent that has been identified as an agent effective to treat cognitive decline using an in vitro screen with an animal and confirming that the test agent is an agent effective to treat cognitive decline in the animal. The confirmation step may be optional. In some embodiments, confirming that the agent is effective in vivo comprises contacting the test agent with an animal model of cognitive decline or an animal model that is associated with a disease that is associated with cognitive decline. In some embodiments, the animal model is a mouse in an Aβ-induced fear condition deficit model. In some embodiments, where the test agent that inhibits the effect seen in the animal model in the absence of the test agent when compared to the presence of the test agent, the test agent is said to be confirmed as an agent effective to treat cognitive decline or associated disease thereof (e.g. Alzheimer's Disease).

Any amyloid β induced fear condition deficit model can be used. As a non-limiting example, the following model and description may be used. The formation of contextual memories is dependent upon the integrity of medial temporal lobe structures such as the hippocampus. In some embodiments of the model, mice are trained to remember that a particular salient context (conditioned stimulus; CS) is associated with an aversive event, for example, a mild foot shock (the unconditioned stimulus, US). Animals that show good learning will express an increase in freezing behavior when placed back into the same context. This freezing is absent in a novel context. Increased freezing in the context indicates a strong hippocampal dependent memory trace in animals.

In some embodiments, three to four month old mice (e.g C57BL/6) from are used. Animals are housed in ventilated cages. Mice may be examined, handled, and weighed prior to initiation of the study to assure adequate health and suitability. In some embodiments, during the course of the study, 12/12 light/dark cycles are maintained. In some embodiments, the room temperature may be maintained between 20 and 23° C. with a relative humidity maintained around 50%. The mice may be single-housed during the entire experimental period. The chow and water may provided ad libitum for the duration of the study. The test may be performed during the animal's light cycle phase.

After anesthesia with, for example, 20 mg/kg Avertin, mice may be implanted with a 26-gauge guide cannula into the dorsal part of the hippocampi (coordinates: posterior=2.46 mm, lateral=1.50 mm to a depth of 1.30 mm). The cannulas may be fixed to the skull with acrylic dental cement (Paladur). After 6-8 d, bilaterally injections of Abeta or vehicle in a final volume of 1 uL over 1 min (200 nM) may be done through infusion cannulas that may be connected to a microsyringe by a polyethylene tube. For fear conditioning, mice may receive a single injection 20 min before the training. Mice may be handled once a day for 3 days before behavioral experiments. During infusion, animals may be handled gently to minimize stress. After infusion, the needle may be left in place for another minute to allow diffusion. After behavioral testing, a solution of 4% methylene blue may be infused into the cannulas. Animals may be sacrificed and their brains may be removed, frozen, and then cut at −20° with a cryostat for histological localization of infusion cannulas.

In some embodiments, for fear conditioning, mice may be placed in a conditioning chamber for 2 min before the onset of a tone [conditioned stimulus (CS)] (e.g. a 30 s, 85 dB sound at 2800 Hz). In the last 2 s of the CS, mice may be given a 2 s, 0.45 mA foot shock (unconditioned stimulus) through the bars of the floor. Then, the mice are left in the conditioning chamber for another 30 s. Freezing behavior, defined as the absence of movement except for that needed for breathing, may be scored using Freezeview software. Contextual fear learning may be evaluated 24 h after training by measuring freezing for 5 min in the chamber in which the mice are trained. Sensory perception of the shock may be determined through threshold assessment. Briefly, the electric current (0.1 mA for 1 s) may be increased at 30 s intervals by 0.1-0.7 mA. Threshold to flinching (first visible response to shock), jumping (first extreme motor response), and vocalization (first vocalized distress) may be quantified for each animal by averaging of the shock intensity at which each animal manifests a behavioral response of that type to the foot shock. Differences in the sensory threshold assessment among different groups of mice in experiments in which fear conditioning is tested is recorded. Moreover, different groups of mice may be tested for a similar exploratory behavior, as demonstrated by a similar percentage of time spent in the center compartment and the number.

In some embodiment, a group of mice (more than one) may be tested per each condition. Compound concentrations, and routes of administration (infusion through cannulas, or gavage, or ip) can be determined by one of skill in the art. Agents that reduce the processed product's effect in the animal model are said to be confirmed.

As used herein, cognitive decline can be any change in an animal's cognitive function. For example cognitive decline, includes but is not limited to, memory loss (e.g. behavioral memory loss), failure to acquire new memories, confusion, impaired judgment, personality changes, disorientation, or any combination thereof. An agent that is effective to treat cognitive decline can also be effective for restoration of long term neuronal potentiation; inhibiting, treating, and/or abatement of neurodegeneration; inhibiting, treating, and/or abatement of general amyloidosis; inhibiting, treating, abatement of one or more of amyloid production, amyloid assembly, amyloid aggregation, amyloid oligomer binding, and amyloid deposition; inhibiting, treating, and/or abatement of the activity/effect of one or more of Abeta oligomers on a neuron cell; and any combination thereof. Cognitive decline or disorder also include but are not limited to dementia, including but not limited to Alzheimer's Disease (AD), Down syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND), Frontotemporal dementia Parkinson's Type (FTDP), progressive supranuclear palsy (PSP), Pick's Disease, Niemann-Pick's Disease, corticobasal degeneration, traumatic brain injury (TBI), dementia pugilistica, Creutzfeld-Jacob Disease and prion diseases; Cognitive Dysfunction in Schizophrenia (CDS); Mild Cognitive Impairment (MCI); Age-Associated Memory Impairment (AAMI); Age-Related Cognitive Decline (ARCD) or preclinical Alzheimer's Disease (PCAD); Cognitive Impairment No Dementia (CIND); Addictions such as nicotine addiction; and the like.

The present invention also provides methods of identifying an agent effective to treat a neurodegenerative or psychiatric disease, wherein a symptom of the disease is cognitive decline. The methods comprises the steps described herein, wherein the agent that is identified is an agent effective to treat the neurodegenerative or psychiatric disease. In some embodiments, the neurodegenerative disease is Alzheimer's Disease.

The present invention also provides for agents identified by the methods described herein. In some embodiments, the present invention also provides pharmaceutical compositions comprising the agents identified by the methods described herein. In some embodiments, the present invention provides methods of treatment. In some embodiments, the method comprises a method of treating cognitive decline or a disease associated therewith comprising administering an agent identified using a method described herein to an animal. In some embodiments, the animal is in need of being treated for cognitive decline or a disease associated therewith.

The present invention also provides for kits. The kits may comprises reagents and/or instructions for performing a method of identifying an agent effective to treat cognitive decline or a disease associated therewith. In some embodiments, the kit comprises a test agent, a positive control, a negative control, or any combination thereof. In some embodiments, the test agent is a library of test agents. In some embodiments, the kit comprises memantine. In some embodiments, the kit comprises a processed product of amyloid precursor protein. In some embodiments, a processed product of amyloid precursor protein is Amyloid β. In some embodiments, the Amyloid β is as described herein. The kit can also comprise instructions comprising instructions on how to perform the method. In some embodiments, the kit comprises an animal model. The animal model can be used to confirm the effectiveness of the agent that is identified to be effective for treating cognitive decline and diseases associated therewith (e.g. neurodegenerative and psychiatric, such as Alzheimer's Disease).

The test agent and/or the processed product of amyloid precursor protein can be dissolved or contained in an aqueous solution. The aqueous solution of the composition may include any number of additional components including, for example, one or more compounds or compositions for adjusting pH of the compositions, one or more compounds or compositions for adjusting salt concentrations, one or more compounds or compositions for stabilizing the test agent and/or the processed product of amyloid precursor protein such as, but not limited to, protease inhibitors, buffers, chelating agents, metals, metal salts, and the like. Such additional components are well known in the art, and any known components may be used in various embodiments. For example, in some embodiments, the pH of the test agent and/or the processed product of amyloid precursor protein may be from about 6.5 to about 8.0 or about 7.0 to about 7.5, and any buffer, acid, or base may be added to the composition to achieve the appropriate pH. In other embodiments, the salt concentration may be adjusted to appropriate concentrations by adding sodium chloride such that the final concentration is about 0.8% to about 10% or about 0.9%. In still other embodiments, the metal ions may be provided in the mixture by including one or more metals or metal salts including, for example, Cu(II), Zn(II), Fe (II), Fe(III), Mg(II), and the like. Without wishing to be bound by theory, the inclusion of metal ions in the solution may enhance oligomerization and/or stabilize amyloid β oligomers.

The amyloid β oligomers of embodiments may be composed of any number of amyloid β monomers consistent with the commonly used definition of “oligomer.” For example, in some embodiments, amyloid β oligomers may include from 2 to about 200 amyloid β monomers, and in other embodiments, amyloid β oligomers may be composed from about 2 to about 150, about 2 to about 100, about 2 to about 50, or about 2 to about 25, amyloid β monomers. The amyloid β oligomers of various embodiments may be distinguished from amyloid β fibrils and amyloid β protofibrils based on the confirmation of the monomers. In particular, the amyloid β monomers of amyloid β oligomers are generally globular consisting of β-pleated sheets whereas secondary structure of the amyloid β monomers of fibrils and protofibrils is parallel β-sheets.

The concentration of amyloid β monomer and/or oligomer in the composition may vary among embodiments and may vary depending on the use of the composition. For example in some embodiments, about 0.1% to about 75%, about 0.5% to about 65% or about 1% to about 50% of the protein components of the composition may be amyloid β monomer or amyloid β oligomer or a combination of amyloid β monomer and oligomer. In other embodiments, the concentration of amyloid β monomer and/or oligomer may be from about 1 pM to about 25 mM, about 50 pM to 20 mM, about 1 nM to about 15 mM, or about 0.5 μM to about 10 mM. In some embodiments, the concentration of the amyloid β monomer and/or oligomer may be from about 10 to 100 fM, 10 to 50 fM, or about 10 to 25 fM.

Without wishing to be bound by theory, amyloid β monomers and/or oligomers may begin self-assembly into larger globular oligomers spontaneously upon being added to the test agent, culture medium or the cell. Therefore, amyloid β may include a combination of monomers, oligomers having various numbers of subunits. The composition of some embodiments, may further include amyloid β protofibrils and amyloid β fibrils which spontaneously form or that were added as part of the amyloid β contacted with the cell. In various embodiments, the concentration of amyloid β monomers and oligomers, and/or in some cases, protofibrils and fibrils, in the composition may vary from the concentration of these elements in the constituent parts and throughout the lifetime of the composition. These species may be in dynamic equilibrium with one another as oligomers, protofibrils, and fibrils may dissociate into monomers which can than reform as new species of oligomers, protofibrils, and fibrils.

The present invention, also provides for methods of identifying compounds that affect amyloid β monomers, amyloid β oligomers, and/or amyloid β fibrils assembly or degrade amyloid β oligomers and/or amyloid β fibrils. For example in addition to the steps described herein, in some embodiments, the methods further comprises measuring amyloid β assembly. Agents used in the presently described methods that inhibit assembly in the presence of a cell can be said to be effective to treat cognitive decline or diseases associated with the same. For example, the test agent can be contacted with the cell prior to an Amyloid β monomer and/or oligomer is contacted with the cell. After the test agent is contacted with the cell the Amyloid β monomer and/or oligomer is contacted with the cell and oligomer assembly is measured. After allowing amyloid β oligomers and/or amyloid β fibrils to assemble in the solution, the solution may be tested to determine whether amyloid β oligomers and/or amyloid β fibrils have formed using, for example, immunoprecipitation of amyloid β oligomers and/or amyloid β fibrils and mass spectroscopy or SDS-PAGE electrophoresis. Compounds that reduce or eliminate amyloid oligomer and/or amyloid fibril formation, or diminish the size of amyloid β oligomers and/or amyloid β fibrils, in the solution as compared to amyloid β oligomers and/or amyloid β fibrils formed in the absence of the test compound may be further characterized for their anti-amyloid β activity as potential treatments for amyloidosis and be said to be effective for the treatment of cognitive decline.

Embodiments are not limited by the type or number of agents that can be tested using the methods described herein. For example, in some embodiments, the test agents may be small molecules, and in certain embodiments, the test agents may be small molecules that are designed to bind to amyloid β monomers, amyloid β oligomers and/or amyloid β fibrils, or proteins that associated with amyloid β monomers, amyloid β oligomers, and/or amyloid β fibrils and may reduce formation of amyloid β fibrils, inhibit growth of amyloid β oligomers and/or amyloid β fibrils, or reduce the size or eliminate preformed amyloid β oligomers and/or amyloid fibrils. In some embodiments, a test agent may be a protein or other biological molecule that interacts with amyloid β monomers, amyloid β oligomers and/or amyloid β fibrils or proteins that associated with amyloid β monomers, amyloid β oligomers and/or amyloid β fibrils and may reduce formation of amyloid β oligomers and/or amyloid β fibrils, inhibit growth of amyloid β oligomers and/or amyloid β fibrils, or reduce the size or eliminate preformed amyloid β oligomers and/or amyloid β fibrils. In still other embodiments, the test agent may be a combination of compounds with known anti-amyloid activity, a combination of test compounds, or a combination of one or more compounds with known anti-amyloid β activity and one or more test compounds. In such embodiments, test compounds, combinations of test compounds, and combinations of compounds with known anti-amyloid β activity and test compounds that exhibit anti-amyloid β activity or improved anti-amyloid β activity may be further characterized for in vivo activity and further developed into anti-amyloidosis pharmaceuticals.

The present invention also provides for methods of diagnosing an animal with cognitive decline or a disease associated therewith, such as Alzheimer's Disease. In some embodiments, the methods of diagnosing an animal with cognitive decline or a disease associated therewith comprises contacting a sample from an individual with a composition comprising at least one neuronal cell; measuring a membrane trafficking effect of the cell, wherein a change in membrane trafficking indicates that the animal has cognitive decline or a disease associated therewith. In some embodiments, the diagnosis indicates that the animal has Alzheimer's Disease.

The sample can be any sample taken from an animal. In some embodiments, the sample is cerebrospinal fluid (CSF). In some embodiments, the sample is blood. In some embodiments, the sample is plasma. In some embodiments, sample is a cellular or tissue extract. For example, a sample from an animal's brain may be extracted and tested in the diagnostic assay. After being extracted the brain sample may, for example, be homogenized. In some embodiments, the brain sample may be used to prepare a cellular extract. In some embodiments, the sample may be cultured prior to preparing the extract. In some embodiments, the sample may be cultured in a cell culture media and the media may be used as the sample. Other types of samples can be tested to determine whether or not the animal has cognitive decline or a disease associated therewith. In some embodiments, the sample comprises an amyloid β monomer, oligomer, or combinations thereof.

In some embodiments, the animal is diagnosed as having cognitive decline or a disease associated therewith wherein after contacting the sample with at least one neuronal cell there is a change in membrane trafficking. The change in membrane trafficking may a change (e.g. increase or decrease) in the rate of endocytosis and/or exocytosis. In some embodiments the rate of endocytosis or exocytosis of a yellow tetrazolium salt and/or reduced formazan is changed. In some embodiments, the rate of exocytosis of a tetrazolium salt is increased or decreased. In some embodiments, the rate of exocytosis or endocytosis of reduced formazan is increased or decreased.

In some embodiments, the animal is diagnosed with cognitive decline or a disease associated therewith when the change in membrane trafficking is at least 1.5 fold, 2 fold, 3 fold, 4 fold, 5 fold, or 10 fold greater in the presence of the sample when compared to a negative control or in the absence of the sample.

In some embodiments, the animal that is being diagnosed is suspected of h of having cognitive decline or a disease associated therewith. In some embodiments the animal that is suspected is a human. In some embodiments, the animal has no symptoms of cognitive decline or diseases associated therewith.

The present invention also provides for kits for diagnosing an animal with cognitive decline or a disease associated therewith. In some embodiments, the kit comprises at least one of instructions for diagnosing said animal; a negative control; a positive control; a neuronal cell; or a yellow tetrazolium salt. In some embodiments, the negative control is a sample from an animal that is not afflicted with cognitive decline or a disease associated therewith. In some embodiments, the sample is blood, plasma, spinal fluid or cerebral spinal fluid. In some embodiments, the kit is used to diagnose Alzheimer's Disease. In some embodiments, the positive control is a sample from an animal afflicted with cognitive decline or a disease associated therewith. In some embodiments, the positive control is memantine or Compound Z.

As stated herein, the kit may comprise a neuronal cell. In some embodiments, the neuronal cell is a primary neuronal cell. In some embodiments, the neuronal cell is an immortalized or transformed neuronal cell. A primary neuronal cell is a neuronal cell that can differentiate into other types of neuronal cells, such as glia cells. In some embodiments, the kit comprises a composition comprising at least one neuronal cell. In some embodiments, the composition comprising at least one neuronal cell is free of glia cells. In some embodiments, the composition comprises less than about 30%, 25%, 20%, 15%, 10%, 5%, or 1% of glia cells. The primary neuronal cell can be derived from any area of the brain of an animal. In some embodiments, the neuronal cell is a hippocampal cell. The presence of glia cells can be determined by any method. In some embodiments, glia cells are detected by the presence of GFAP and neurons can be detected by staining positively with antibodies directed against MAP2.

The present invention also provides methods of identifying an Abeta binding partner. In some embodiments, the method comprises contacting an Abeta monomer, oligomer, or combination thereof with at least one neuronal cell comprising a test binding partner and determining the rate of membrane trafficking in the cell, wherein a change in the rate of membrane trafficking indicates that the binding partner is an Abeta binding partner. In some embodiments, when the Abeta monomer, oligomer, or combination thereof is contacted with the cell that does not contain the test binding partner there is no effect on the membrane trafficking in the cell. Therefore, in some embodiments, a change is only seen in membrane trafficking in the presence of the test binding partner, which indicates that the test binding partner is a binding partner of an Abeta monomer, oligomer, or combination thereof.

In some embodiments, the test binding partner is exogenously expressed. In some embodiments, the neuronal cell is transfected, infected, or transformed with said test binding partner. In some embodiments, the test binding partner is a receptor. In some embodiments, the method further comprises contacting the cell with a pathway inhibitor. A pathway inhibitor, can be for example, a compound or reagent that inhibits specific pathways within the cells. For example there are compounds and reagents that can inhibit receptor tyrosine kinase activity, GPCR activity and the like. In some embodiments, the pathway inhibitor is a g-protein inhibitor, a kinase inhibitor, or a phosphatase inhibitor. In some embodiments, the kinase inhibitor is a tyrosine kinase inhibitor, serine or threonine kinase inhibitor, or a PI-3 kinase inhibitor. In some embodiments, the inhibitor inhibits an Abeta-mediated effect on the cell. The change in membrane trafficking can be determined as described throughout the present specification, including but not limited to, the MTT assay.

### EXAMPLES

The invention is now described with reference to the following examples. These examples are provided for the purpose of illustration only and the invention should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### Example 1

MTT assay Primary neurons from E18 Sprague-Dawley rat embryos were plated at optimized concentrations in 384 well plates in NB media (Invitrogen). Neurons were maintained in cultures for 3 weeks, with twice weekly feeding of NB media with N2 supplement (Invitrogen). Vehicle or Abeta oligomer preparations (1.5 uM), followed by compounds were added to cells and incubated for 1 to 24 hr at 37° C. in 5% CO₂. MTT reagent (3-(4,5-dimethylthizaol-2-yl)-2,5diphenyl tetrazolium bromide) reagent (Roche Molecular Biochemicals) was reconstituted in phosphate buffered saline to 5 mg/ml. 10 ul of MTT labeling reagent was added to each well and incubated at 37° C. for 1 h, then imaged.

Each assay plate was formatted so that compounds were tested with and without Abeta on each plate. This enables the experiments to eliminate toxic or metabolically active compounds early on in the screening cascade (at the level of the primary screen).

Experimental controls: Abeta 1-42 oligomers were made and used as positive controls. The oligomers can be made by any method, such as published methods (Dhalgren et al., '02, LeVine '04, Shrestha et al., '06, Puzzo et al., '05, Barghorn et al., '05, Johansson et al., '06). Abeta 1-42 oligomers were also isolated from human brain tissue (Walsh et al., '02, Lesne et al., '06, Shankar et al '08) and acted as the positive controls. Negative controls included vehicle-treated neurons as well as neurons treated with Compound Z. These controls, on each plate, serve as normalization tools to calibrate assay performance on a plate-by-plate basis. This yields confidence in inhibition results, but and allows one to track the assay's performance and monitor for potential systematic variability over time.

Statistical software and Analysis: Data handling and analysis was accomplished by Cellomics VTI image analysis software and STORE automated database software. Statistical comparisons were made via pairwise Tukey-Kramer analysis to determine the significance of the separation between compound+Abeta oligomers from Abeta alone, and between compound alone from vehicle. The ability of mature primary neurons to more closely approximate the electrophysiologically mediated signal transduction network of the adult brain justified this screening method. Power analysis was set for a number of replicate screening wells that will minimize false negatives such as N=4. Rank ordering of compounds was done on the basis of secondary assay mechanism of action and physicochemical properties of the hit structures.

A beta oligomer preparations: Human amyloid peptide 1-42 was obtained from a variety of commercial sources, with lot-choice contingent upon quality control analysis. Abeta 1-42 oligomers made according to published methods (Dhalgren et al., '02, LeVine '04, Shrestha et al., '06, Puzzo et al., '05, Barghorn et al., '05, Johansson et al., '06) or isolated from human brain tissue (Walsh et al., '02, Lesne et al., '06, Shankar et al '08) served as positive controls. Toxicity was monitored in each image-based assay via quantification of nuclear morphology visualized with the DNA binding dye DAPI (Invitrogen). Nuclei that were fragmented were considered to be in late stage apoptosis (Majno and Joris '95). Peptide lots producing unusual peptide size ranges or significant toxicity at standard concentrations on neurons were rejected.

Image Processing: Images are captured and analyzed with the Cellomics VTI automated microscope platform. For statistical analysis, a Tukey-Kramer pair-wise comparison with unequal variance is used.

### Example 2

### A Primary Neuron-Based Functional Screening Assay to Detect Small Molecule Abeta Oligomer Blockers

Primary rat neurons grown for at least 3 weeks in vitro were chosen as the basis for this screening assay. These neurons express the full complement of synaptic proteins characteristic of neurons in the mature brain, and exhibit a complex network of activity-dependent electrical signaling. Neurons and glia in such cultures have molecular signaling networks exhibiting excellent registration with intact brain circuitry, and for this reason have been used for over two decades as a model system for learning and memory (See e.g. Kaech S, Banker G. Culturing hippocampal neurons. Nat. Protoc. 2006; 1(5):2406-15. Epub 2007 Jan. 11; See also Craig A M, Graf E R, Linhoff M W. How to build a central synapse: clues from cell culture. Trends Neurosci. 2006 January; 29 (1):8-20. Epub 2005 Dec. 7. Review). More complex systems such as acute or organotypic brain slices are very useful but not amenable to high throughput screening. Immortalized or transformed neuronal cell lines are amenable to high throughput screening, but do not replicate the electrophysiological state-dependent signaling of primary neuronal cultures and are unlikely to adequately model the subtle alterations in this signaling that are caused by oligomers during the earliest manifestations of the disease state (See e.g. Görtz P, Fleischer W, Rosenbaum C, Otto F, Siebler M. Neuronal network properties of human teratocarcinoma cell line-derived neurons. Brain Res. 2004 Aug. 20; 1018(1):18-25). For this reason, primary neuronal cultures were chosen because of their ability to be used in high throughput screens and fidelity to what occurs in vivo.

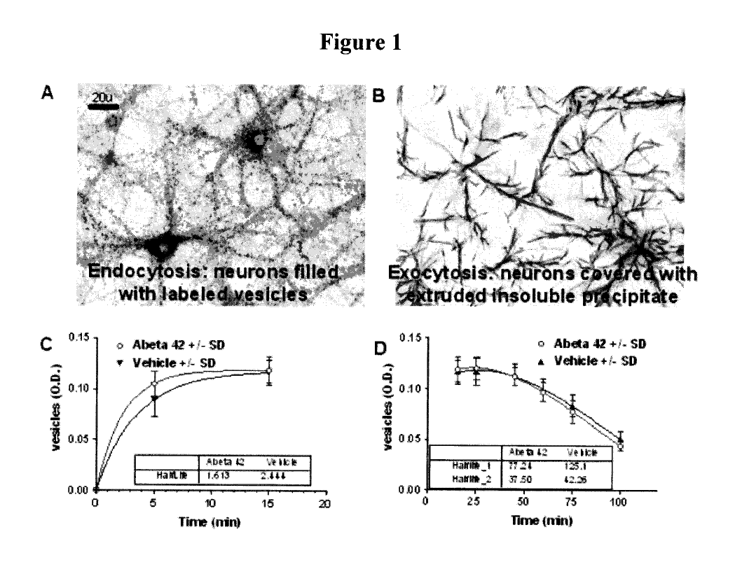

Reduced formazan was first visible in intracellular vesicles (FIG. 1A). Example of neurons filled with labeled vesicles following endocytosis of dye and reduction to an insoluble purple product. (Scale bar=20 microns in FIG. 1A). Eventual formazan exocytosis was accelerated via Abeta oligomers in mature hippocampal neurons in vitro (FIG. 1B). Example photomicrograph of neurons covered with insoluble purple dye that have been extruded via exocytosis. The dye precipitated in the aqueous environment of the culture and formed needle-shaped crystals on the surface of the neuron. (FIG. 1B). Endocytosis rate was altered in the presence of Abeta oligomers. (FIG. 1C) Exocytosis rate was altered in the presence of Abeta oligomers (FIG. 1D).

Since synaptic and memory deficits, and not widespread cell death, predominate at the earliest stages of Alzheimer's Disease, assays that measure these changes can be used to discover small molecule inhibitors of oligomer activity. The MTT assay, such as that of Example 1 herein, can be used as a measure of toxicity in cultures. Yellow tetrazolium salts were endocytosed by cells and reduced to insoluble purple formazan in the endosomal pathway. The level of purple formazan was a reflection of the number of actively metabolizing cells in culture, and reduction in the amount of formazan was taken as a measure of cell death or metabolic toxicity in culture. When observed through a microscope, the purple formazan was first visible in intracellular vesicles that fill the cell (FIG. 1A). Over time, the vesicles were exocytosed and the formazan precipitated as needle-shaped crystals on the outer surface of the plasma membrane as the insoluble formazan was exposed to the aqueous media environment (FIG. 1B). Cells respond to sublethal levels of Abeta oligomers by selectively accelerating the exocytosis rate of reduced formazan, while leaving endocytosis rate unaffected, which can be seen in mature primary neurons in vitro and quantified these morphological shifts via automated microscopy and image processing. At a given point in time following tetrazolium salt addition to the culture well, vehicle-treated cells had the appearance of those in FIG. 1A, while Abeta oligomer-treated cells had the appearance of those in FIG. 1B. Under these circumstances, there was no overall change in the total amount of reduced formazan, simply a shift in its morphology. This assay is sensitive to low levels of oligomers that do not cause cell death.

Evidence suggests that Abeta oligomer-mediated reduction in neuronal surface receptor expression mediated by membrane trafficking are the basis for oligomer inhibition of electrophysiological measures of synaptic plasticity (LTP) and thus learning and memory (See Kamenetz F, Tomita T, Hsieh H, Seabrook G, Borchelt D, Iwatsubo T, Sisodia S, Malinow R. APP processing and synaptic function. Neuron. 2003 Mar. 27; 37 (6):925-37; and Hsieh H, Boehm J, Sato C, Iwatsubo T, Tomita T, Sisodia S, Malinow R. AMPAR removal underlies Abeta-induced synaptic depression and dendritic spine loss. Neuron. 2006 Dec. 7; 52(5):831-43). Measuring membrane trafficking rate changes induced by oligomers via formazan morphological shifts has been used in cell lines to discover Abeta oligomer-blocking drugs [Maezawa I, Hong H S, Wu H C, Battina S K, Rana S, Iwamoto T, Radke G A, Pettersson E, Martin G M, Hua D H, Jin L W. A novel tricyclic pyrone compound ameliorates cell death associated with intracellular amyloid-beta oligomeric complexes. J. Neurochem. 2006 July; 98(1):57-67; Liu Y, Schubert D. Cytotoxic amyloid peptides inhibit cellular 3-(4,5-dimethylthiazol-2-yl)-2,5-diphenyltetrazolium bromide (MTT) reduction by enhancing MTT formazan exocytosis. J. Neurochem. 1997 December; 69(6):2285-93; Liu Y, Dargusch R, Banh C, Miller C A, Schubert D. Detecting bioactive amyloid beta peptide species in Alzheimer's Disease. J. Neurochem. 2004 November; 91 (3):648-56; Liu Y, Schubert D. Treating Alzheimer's Disease by inactivating bioactive amyloid beta peptide. Curr Alzheimer Res. 2006 April; 3(2):129-35; Rana S, Hong H S, Barrigan L, Jin L W, Hua D H. Syntheses of tricyclic pyrones and pyridinones and protection of Abeta-peptide induced MC65 neuronal cell death. Bioorg Med Chem. Lett. 2009 Feb. 1; 19(3):670-4. Epub 2008 Dec. 24; and Hong H S, Maezawa I, Budamagunta M, Rana S, Shi A, Vassar R, Liu R, Lam K S, Cheng R H, Hua D H, Voss J C, Jin L W. Candidate anti-Abeta fluorene compounds selected from analogs of amyloid imaging agents. Neurobiol Aging. 2008 Nov. 18. (Epub ahead of print)] that lower Abeta brain levels in rodents in vivo [Hong H S, Rana S, Barrigan L, Shi A, Zhang Y, Zhou F, Jin L W, Hua D H. Inhibition of Alzheimer's amyloid toxicity with a tricyclic pyrone molecule in vitro and in vivo. J. Neurochem. 2009 February; 108(4): 1097-1108].

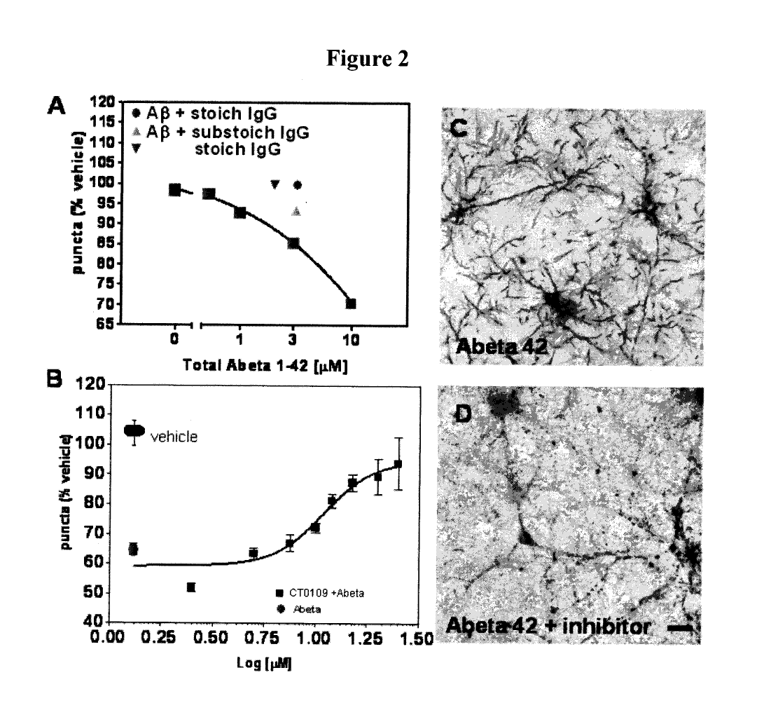

The exocytosis assay was adapted for use with mature primary neuronal cultures grown for 3 weeks in vitro. Abeta oligomers caused a dose-dependent decrease in the amount of intracellular vesicles (puncta) filled with reduced purple formazan (FIG. 2A, squares; 3 μM dose corresponds to image in FIG. 2C) as measured via image processing using a Cellomics VTI automated microscopy system. Increasing the amount of Abeta oligomers eventually resulted in overt toxicity. Thus, the concentration of neuroactive Abeta oligomers was much lower than that causing cell death. This decrease can be blocked by adding stoichiometric amounts of anti-Abeta monoclonal antibody 6E10 (IgG) to the cultures prior to oligomer addition (FIG. 2A, circle; the circle corresponds to image in FIG. 2D; antibody alone [down triangle] has no effect on the neurons). Several compounds were tested that have been reported to block the effects of Abeta oligomers, including the sugar alcohol scyllo-inositol (AZD-103), the nAChR antagonist hexamethonium bromide, and the NMDAR antagonists MK-801 and none were active (Fenili et al., '07, Calabrese et al., '06, LeCor et al., '07).

The assay was optimized for performance in 384-well microtiter plates with automated liquid handling robotics for compound formatting and assay plate stamping, routinely achieving statistically significant two-fold separation between vehicle and Abeta oligomer-treated neurons (Student's t-test, unequal variance). Test Compounds were added to neurons first, then oligomers were added. When configured in this manner the assay was able to detect compounds that act via disruption of oligomers, inhibition of oligomer binding to neurons, and counteraction of signal transduction mechanisms of action initiated by oligomer binding and combinations thereof.

Compounds were considered active if they significantly block Abeta-mediated changes in membrane trafficking, but do not significantly affect membrane trafficking when dosed on their own. An example is shown in FIG. 2B; Compound Z inhibits oligomer effects on membrane trafficking with an EC50 of 7 μM.

FIG. 2A shows dose-dependent decrease of intracellular formazan-filled vesicles (puncta) caused by Abeta 42 oligomer treatment acceleration of exocytosis (squares). Oligomer effects were blocked by anti Abeta IgG (circle and up triangle; circle refers to stoich amount of IgG, i.e., 3 μM of Aβ and 1.5 μM of IgG; up triangle refers to substoich IgG, i.e., 3 μM of Aβ and 0.5 μM of IgG). IgG itself (down triangle) has no effect. FIG. 2B shows Compound Z, which inhibits oligomer effects on membrane trafficking. FIG. 2C shows representative micrographs of 21 DIV hippocampal neurons in vitro showing oligomer effects membrane trafficking (corresponding to data point 3 μM in FIG. 2A); and FIG. 2D shows blockade by anti-Abeta antibodies (corresponding to the circle in FIG. 2A). Data were the average of 3 experiments. Scale bar=20 micron in FIG. 2D.

### Example 3

### Fear Conditioning Assay

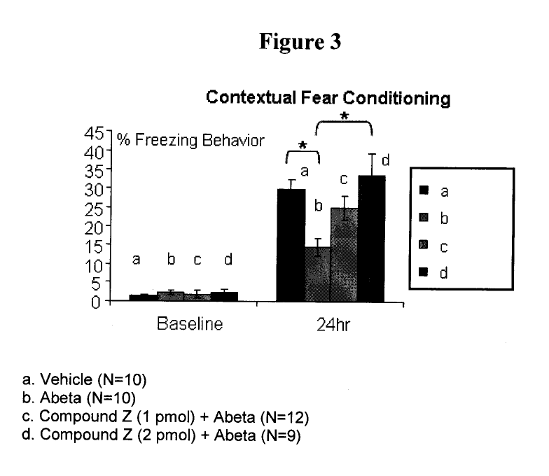

Compound Z was tested in an animal model of a memory-dependent behavioral task known as fear conditioning. The study protocol was designed based on published protocols (See e.g. Puzzo D, Privitera L, Leznik E, Fá M, Staniszewski A, Palmeri A, Arancio O. Picomolar amyloid-beta positively modulates synaptic plasticity and memory in hippocampus. J. Neurosci. 2008 Dec. 31; 28(53):14537-45). The formation of contextual memories is dependent upon the integrity of medial temporal lobe structures such as the hippocampus. In this assay mice were trained to remember that a particular salient context (conditioned stimulus; CS) is associated with an aversive event, in this case a mild foot shock (the unconditioned stimulus, US). Animals that show good learning will express an increase in freezing behavior when placed back into the same context. This freezing is absent in a novel context. Increased freezing in the context indicates strong hippocampal-dependent memory formation in animals. Memory tested in Fear Conditioning is sensitive to elevations of soluble Aβ. FIG. 3 shows the results of administration of Abeta oligomers (bar labeled with “a”) during training results in memory deficits when animals are tested about 24 hours later, compared to vehicle administration (bar labeled with “b”). Example Compound Z was effective at stopping Abeta oligomer mediated effects on membrane trafficking (FIG. 3). When administered to animals prior to Abeta oligomer administration, Example Compound Z blocked oligomer effects on memory in a dose-dependent manner. The compound completely blocked oligomer-mediated memory deficits at the 2 pmol dose (FIG. 3, bar labeled with “d”). This behavioral efficacy demonstrates that the membrane trafficking assay is able to predict which compounds will be efficacious in treating the behavioral memory loss caused by oligomers. The fear condition model for memory was performed as described herein.

FIG. 3 shows that Abeta produces significant deficits in memory formation vs. vehicle (p<0.05) in the contextual fear conditioning memory task. FIG. 3 shows that the 2 pmol dose of Compound Z+Abeta (200 nM) completely blocked the effect of Abeta on memory (p<0.05, one way ANOVA, post hoc comparison with Bonferroni correction). No effect of compound alone was observed (data not shown). No adverse behavioral changes were observed at any dose.

### Example 4

### Membrane Trafficking Assay

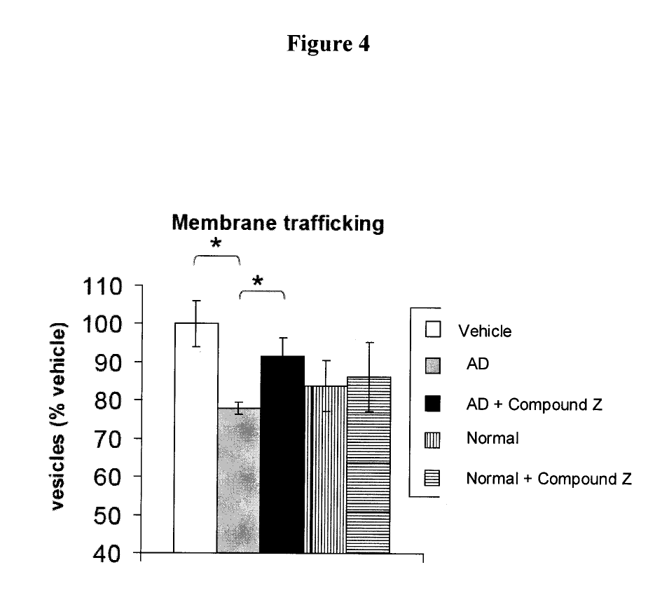

Abeta assemblies were isolated from patients with Alzheimer's Disease (AD) or from normal patients. The Abeta assemblies were tested for their ability to modulate membrane trafficking. HMW (>100 KDa) Abeta assemblies isolated from AD patients do not affect membrane trafficking (not shown). IMW (10-100 KDa) Abeta assemblies isolated from AD patients significantly affect membrane trafficking. (FIG. 4). IMW Abeta assemblies isolated from Age-matched normal individuals do not affect membrane trafficking (FIG. 4). Compound Z has no effect on Abeta assemblies isolated from Age-matched normal individuals. (FIG. 4). Compound Z significantly blocked the trafficking effects of AD-brain derived Abeta assemblies. (FIG. 4). The assay was performed as described herein.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the invention, in addition to those described herein, will be apparent to those skilled in the art from the foregoing description. Such modifications are also intended to fall within the scope of the appended claims.

1. A method of identifying an agent effective to treat cognitive decline, the method comprising the steps of:
contacting a test agent and a processed product of amyloid precursor protein with a composition comprising at least one neuronal cell; and
measuring a processed product of amyloid precursor protein-mediated effect on the at least one neuronal cell, wherein a test agent that inhibits the processed product of amyloid precursor protein-mediated effect on the cell is thereby identified as an agent that is effective to treat cognitive decline.

2. (canceled)

3. The method of claim 1, wherein the processed product of amyloid precursor protein is at least one of an Abeta monomer, Abeta oligomer, or combinations thereof.

4. (canceled)

5. The method of claim 1, wherein the test agent is an agent effective to treat cognitive decline, and wherein the test agent does not significantly affect membrane trafficking in the absence of the Abeta monomer, Abeta oligomer, or combinations thereof.

6. (canceled)

7. (canceled)

8. (canceled)

9. (canceled)

10. The method of claim 1, wherein the processed product of amyloid precursor protein is contacted with the composition comprising at least one neuronal cell at a sublethal concentration.

11. (canceled)

12. The method of claim 1, wherein the at least one neuronal cell is grown for at least 3 weeks in vitro prior to being contacted with test agent.

13. (canceled)

14. The method of claim 2, wherein the effect on membrane trafficking is determined by an MTT assay.

15. The method of claim 1, wherein the step of measuring a processed product of amyloid precursor protein-mediated effect on the composition comprising at least one neuronal cell comprises measuring metabolism of yellow tetrazoilum by the cell.

16. The method of claim 1, wherein the step of measuring a processed product of amyloid precursor protein-mediated effect on the composition comprising at least one neuronal cell comprises visualizing morphology of at least one cell in the composition.

17. (canceled)

18. (canceled)

19. (canceled)

20. (canceled)

21. The method of claim 1 further comprising the step of contacting a test agent identified as an agent effective to treat cognitive decline with an animal, and thereby confirming that the test agent is an agent effective to treat cognitive decline.

22. The method of claim 21, wherein the step of confirming comprises contacting the test agent with a mouse in an Abeta-induced fear condition deficit model, wherein a test agent that inhibits the effects of Abeta in the model is thereby confirmed to be an agent effective to treat cognitive decline.

23. (canceled)

24. (canceled)

25. The method of claim 1, wherein an agent that is effective to treat cognitive decline is an agent that is effective for at least one of:
(i) restoration of long term neuronal potentiation;
(ii) inhibiting, treating, and/or abatement of neurodegeneration;
(iii) inhibiting, treating, and/or abatement of general amyloidosis;
(iv) inhibiting, treating, and/or abatement of one or more of amyloid production, amyloid assembly, amyloid aggregation, amyloid oligomer binding, and amyloid deposition; or
(v) inhibiting, treating, and/or abatement of the activity/effect of one or more of Abeta oligomers on a neuron cell.

26. (canceled)

27. A method of identifying an agent effective to treat a neurodegenerative or psychiatric disease, wherein a symptom of the disease is cognitive decline, the method comprising the steps of: contacting a test agent and a processed product of amyloid precursor protein with a composition comprising at least one neuronal cell; and measuring a processed product of amyloid precursor protein-mediated effect on the at least one neuronal cell, wherein a test agent that inhibits the processed product of amyloid precursor protein-mediated effect on the cell is thereby identified as an agent effective to treat the neurodegenerative or psychiatric disease.

28. (canceled)

29. (canceled)

30. (canceled)

31. (canceled)

32. (canceled)

33. A kit for identifying an agent effective to treat cognitive decline comprising at least one of:
a test agent;
a negative control
a positive control
a neuronal cell
a processed product of amyloid precursor protein; or
instructions on how to identify an agent effective to treat cognitive decline.

34. (canceled)

35. The kit of claim 33, wherein the positive control is memantine.

36. The kit of claim 33, wherein the neuronal cell is selected from a primary neuronal cell and a hippocampal neuron.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. The method of claim 38, wherein the animal has no symptoms of cognitive decline or diseases associated therewith.

49. A kit for diagnosing an animal with cognitive decline or a disease associated therewith comprising at least one of:
instructions for diagnosing said animal;
a negative control;
a positive control;
a neuronal cell; or
a yellow tetrazolium salt.

50. The kit of claim 49, wherein the negative control is a sample from an animal that is not afflicted with cognitive decline or a disease associated therewith.

51. The kit of claim 49, wherein the disease diagnosed is Alzheimer's Disease.

52. The kit of claim 49, wherein the positive control is a sample from an animal afflicted with cognitive decline or a disease associated therewith.

53. (canceled)

54. A method of identifying an Abeta binding partner comprising:
contacting an Abeta monomer, Abeta oligomer, or combination thereof with at least one neuronal cell comprising a test binding partner
determining the rate of membrane trafficking in the cell,
wherein a change in the rate of membrane trafficking indicates the binding partner is an Abeta binding partner.

55. (canceled)

56. (canceled)

57. (canceled)

58. (canceled)

59. (canceled)

60. (canceled)

61. (canceled)

62. (canceled)
